(19) United States
(12) Patent Application Publication
(10) Pub. No.: US 2014/0213483 A1
(43) Pub. Date: Jul. 31, 2014

(54) LABELED DEVICE

(71) Applicants: Uwe Radelof, Potsdam (DE); Joerg Hempel, Schulzendorf (DE); Holger Eickhoff, Berlin (DE)

(72) Inventors: Uwe Radelof, Potsdam (DE); Joerg Hempel, Schulzendorf (DE); Holger Eickhoff, Berlin (DE)

(21) Appl. No.: 14/235,401

(22) PCT Filed: Jul. 27, 2012
(86) PCT No.: PCT/EP2012/003211

§ 371 Date: Apr. 7, 2014

(30) Foreign Application Priority Data
Jul. 27, 2011 (EP) 11006174.4

(51) Int. Cl. C07H 21/04

(52) U.S. Cl. CPC C07H 21/04
USPC 506/16; 506/32

(57) ABSTRACT

A microarray is disclosed comprising a reaction zone and a labeling zone, wherein the at least one labeling zone contains a marker, such as an alphanumerical code, a binary code, a barcode, a 2D code, a colour code or a triplet code and wherein the marker allows the identification of the microarray.

The present invention is related to a device comprising a first surface and a second surface, at least one reaction zone and at least one labeling zone, a method for the manufacture or making of such device.

A microarray is an arrangement of a plurality of reaction zones whereby each reaction zone of said plurality of reaction zones is located at a specific position of a surface thus forming a two-dimensional matrix. As the position of each reaction zone on the surface is known it is possible to correlate a signal which is generated at a distinct position on the surface with an event happening at the reaction zone located at the distinct position. In a microarray the reaction zones differ as to the reaction occurring there and such difference in reaction is caused by the different reagents present in the individual reaction zone. Typically, a first chemical compound is attached to a reaction zone and said first chemical compound is different for a variety of the reaction zones of the microarray. In many microarrays the first chemical compound is different for each reaction zone, although there may be one or more reaction zones in a microarray where the first chemical compound is the same. Microarrays are particularly suitable in high-throughput screening methods.

Depending of the chemical nature of the first chemical compound, various types of microarrays can be distinguished including, but not limited to DNA microarrays, RNA microarrays, peptide microarrays, protein microarrays, antibody microarrays and carbohydrate arrays. There are, however, other ways to categorize microarrays such as depending on the density of the reaction zones of such microarrays as in case of high-density microarrays and low-density microarrays, depending on the kind of agent to be detected or immobilized to the individual reaction zones such in antigen/allergen microarrays, or depending on the intended use of such microarrays such as in case of R&D microarrays and IVD microarrays.

In most cases, individual microarrays are difficult to be distinguished and thus to be identified. Identification, however, is necessary for various reasons such as characterization in terms of the reaction zones and first chemical compounds attached to the reaction zones, production related information such as production date and production lot etc.

The identification of a microarray requires the labeling of the microarray. The prior art suggests various ways to provide such labeling. The simplest but also most error-prone way is to provide such labeling by adding to the microarray a marker which is a hand-written identification code. Such marker or identification code can be a running number or any other code such as a bar code or the like which allows the identification of the thus labeled microarray. Another possibility is that such marker is added to the microarray prior to or after the production step whereby such marker is generated by a device and added to the microarray by said device generating the marker or a device which is different from said device generating the marker.

These approaches of the prior art for the labeling of a microarray suffer from various shortcomings. Adding such marker to the microarray by hand-writing is time consuming and prone to error because of human intervention. Using a device for adding such marker to the microarray in accordance with the technical teaching of the prior art requires additional devices besides those devices used in the preparation of the microarray.

The problem underlying the instant invention is thus the provision of a microarray bearing a marker which allows the identification of the microarray, whereby the adding of the marker requires less time, is less error-prone and requires fewer devices.

A further problem underlying the instant invention is the provision of a method for manufacturing a labeled device, whereby the labeled device comprises a marker which allows the identification of the device.

A still further problem underlying the instant invention is the provision of a method for marking a device such that the device may be identified.

These and other problems are solved by the subject matter of the attached independent claims. Preferred embodiments may be taken from the dependent claims. It will be understood by a person skilled in the art that the embodiments of the labeled device according to the present invention described herein are also embodiments of the method according to the present invention and vice versa.

More specifically, the problem underlying the present invention is solved in a first aspect which is also the first embodiment of the first aspect, by a labeled device comprising a first surface and a second surface, at least one reaction zone and at least one labeling zone, wherein

the at least one reaction zone is arranged on the first or the second surface and the at least one labeling zone is arranged on the first or the second surface,

the at least one reaction zone allows performing a reaction, preferably a chemical, physical, biochemical or biological reaction,

the at least one labeling zone contains a marker, wherein the marker allows the identification of the device and wherein

the labeled device is obtained by a method for the preparation of the labeled device, wherein

a first chemical compound is added to the at least one reaction zone using a first delivery device,

the marker is added to the at least one labeling zone prior, during or after the adding of the first chemical compound to the at least one labeling zone using a second delivery device,

wherein the at least one reaction zone is arranged in a plane forming a reaction plane, and the at least one labeling zone is arranged in a plane forming a labeling plane and wherein the reaction plane and the labeling plane are at least partially overlapping.

In a second embodiment of the first aspect which is also an embodiment of the first embodiment of the first aspect, the at least one reaction zone and the at least one labeling zone are arranged on the first surface.

In a third embodiment of the first aspect which is also an embodiment of the first embodiment of the first aspect, the at least one reaction zone is arranged on the first surface and the at least one labeling zone is arranged on the second surface.

In a fourth embodiment of the first aspect which is also an embodiment of the first, the second and the third embodiment of the first aspect, the at least one reaction zone and the at least on labeling zone are overlapping or are at least partially overlapping.

In a fifth embodiment of the first aspect which is also an embodiment of the first, the second and the third embodiment of the first aspect, the at least one reaction zone and the at least one labeling zone are not overlapping.

In a sixth embodiment of the first aspect which is also an embodiment of the first, the second, the third, the fourth and the fifth embodiment of the first aspect, the marker and the at least one reaction zone form an identification entity.

In a seventh embodiment of the first aspect which is also an embodiment of the first, the second, the third, the fourth, the fifth and the sixth embodiment of the first aspect, the reaction plane and the labeling plane are detectable at the same time.

In an eighth embodiment of the first aspect which is also an embodiment of the first, the second, the third, the fourth, the fifth, the sixth and the seventh embodiment of the first aspect, the reaction plane is detected by a first detection device and the labeling plane is detected by a second detection device, preferably the reaction plane and the labeling plane are detected by the same detection device.

In a ninth embodiment of the first aspect which is also an embodiment of the eighth embodiment of the first aspect, the reaction plane and the detection plane are arranged in a depth of focus of the detection device.

In a tenth embodiment of the first aspect which is also an embodiment of the eighth and the ninth embodiment of the first aspect, the first detection device or the same detection device is monitoring a reaction or is detecting the result of a reaction in the at least one reaction zone.

In an eleventh embodiment of the first aspect which is also an embodiment of the first, the second, the third, the fourth, the fifth, the sixth, the seventh, the eighth, the ninth and the tenth embodiment of the first aspect, the reaction is an interaction between at least a first chemical compound and a second chemical compound.

In a twelfth embodiment of the first aspect which is also an embodiment of the eleventh embodiment of the first aspect, the first chemical compound is attached to the at least one reaction zone and the second chemical compound is contained in a fluid arranged at the at least one reaction zone.

In a 13th embodiment of the first aspect which is also an embodiment of the twelfth embodiment of the first aspect, the fluid is covering the at least one reaction zone or part thereof.

In a 14th embodiment of the first aspect which is also an embodiment of the eleventh, the twelfth and the 13th embodiment of the first aspect, the first chemical compound is selected from the group comprising nucleic acid molecules, peptides, polypeptides and proteins.

In a 15th embodiment of the first aspect which is also an embodiment of the first, the second, the third, the fourth, the fifth, the sixth, the seventh, the eighth, the ninth, the tenth, the eleventh, the twelfth, the 13th and the 14th embodiment of the first aspect, the marker allows or is suitable for identification of the device after the reaction has been performed.

In a 16th embodiment of the first aspect which is also an embodiment of the first, the second, the third, the fourth, the fifth, the sixth, the seventh, the eighth, the ninth, the tenth, the eleventh, the twelfth, the 13th, the 14th and the 15th embodiment of the first aspect, the marker is an alphanumerical code, a binary code, a bar code, a 2D code, a color code, a triplet code or a combination thereof.

In a 17th embodiment of the first aspect which is also an embodiment of the first, the second, the third, the fourth, the fifth, the sixth, the seventh, the eighth, the ninth, the tenth, the eleventh, the twelfth, the 13th, the 14th, the 15th and the 16th embodiment of the first aspect, the marker is formed by an ink, a dye or a biological compound.

In a 18th embodiment of the first aspect which is also an embodiment of the 17th embodiment of the first aspect, the biological compound is selected from the group comprising a nucleic acid molecule, an oligonucleotide, a DNA oligonucleotide, an RNA oligonucleotide, an LNA oligonucleotide, a biotinylated oligonucleotide, a biotinylated DNA oligonucleotide, a biotinylated RNA oligonucleotide, a biotinylated LNA oligonucleotide, a color-labeled oligonucleotide, a color-labeled DNA oligonucleotide, a color-labeled RNA oligonucleotide, a color-labeled LNA oligonucleotide, a protein, an oligopeptide, a peptide, an antibody, a biotinylated protein, a biotinylated oligopeptide, a biotinylated peptide, a biotinylated antibody.

In a 19th embodiment of the first aspect which is also an embodiment of the 17th and the 18th embodiment of the first aspect, the marker is formed by a nucleic acid.

In a 20th embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th and the 19th embodiment of the first aspect, preferably of the 17th, the 18th and the 19th embodiment of the first aspect, the marker is detectable upon completion of the reaction on the at least one reaction zone.

In a 21st embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th and the 19th embodiment of the first aspect, preferably any one of the 17th and the 18th embodiment of the first aspect, the marker becomes detectable when the reaction on the at least one reaction zone is performed.

In a 22nd embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th and the 21st embodiment of the first aspect, the device comprises a plurality of reaction zones.

In a 23rd embodiment of the first aspect which is also an embodiment of the 22nd embodiment of the first aspect, at least a part of the plurality of reaction zones are arranged in the reaction plane, preferably all reaction zones of the plurality of reaction zones are arranged in the reaction plane.

In a 24th embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th, the 21st, the 22nd and the 23rd embodiment of the first aspect, the device is a slide and wherein the slide contains the plurality of reaction zones.

In a 25th embodiment of the first aspect which is also an embodiment of the 24th embodiment of the first aspect, the reaction zones are each and individually a hydrophilic anchor surrounded by a hydrophobic zone.

In a 26th embodiment of the first aspect which is also an embodiment of the 24th and the 25th embodiment of the first aspect, wherein the reaction is a hybridization reaction, wherein different nucleic acid molecule species are attached to various reaction zones of the plurality of reaction zones, and wherein each reaction zone contains a single nucleic acid molecule species.

In a 27th embodiment of the first aspect which is also an embodiment of the 24th and the 25th embodiment of the first aspect, the reaction is an antigen-antibody reaction wherein different antibody species are attached to various reaction zones of the plurality of reaction zones, and each reaction zone contains a single antibody species.

In a 28th embodiment of the first aspect which is also an embodiment of the 24th and the 25th embodiment of the first aspect, the reaction is an antigen-antibody reaction wherein different antigen species are attached to various reaction zones of the plurality of reaction zones, and each reaction zone contains a single antigen species.

In a 29th embodiment of the first aspect which is also an embodiment of the 24th, the 25th, the 26th, the 27th and the 28th embodiment of the first aspect, the marker is formed by a nucleic acid molecule.

In a 30th embodiment of the first aspect which is also an embodiment of the 24th, the 25th, the 26th, the 27th, the 28th and the 29th embodiment of the first aspect, the labeling zone and the reaction zones are arranged on the first surface.

In a 31st embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th, the 21st, the 22nd and the 23rd embodiment of the first aspect, the device is a multi-well plate comprising a plurality of wells which are connected through links, fixed links or fins.

In a 32nd embodiment of the first aspect which is also an embodiment of the 31st embodiment of the first aspect, at least one of the wells contains the plurality of reaction zones.

In a 33rd embodiment of the first aspect which is also an embodiment of the 32nd embodiment of the first aspect, the reaction zones are each and individually a hydrophilic anchor surrounded by a hydrophobic zone.

In a 34th embodiment of the first aspect which is also an embodiment of the 31st, the 32nd and the 33rd embodiment of the first aspect, the reaction is a hybridization reaction, wherein different nucleic acid molecule species are attached to various reaction zones of the plurality of reaction zones, and wherein each reaction zone contains a single nucleic acid molecule species.

In a 35th embodiment of the first aspect which is also an embodiment of the 31st, the 32nd and the 33rd embodiment of the first aspect, the reaction is an antigen-antibody reaction wherein different antibody species are attached to various reaction zones of the plurality of reaction zones, and each reaction zone contains a single antibody species.

In a 36th embodiment of the first aspect which is also an embodiment of the 31st, the 32nd and the 33rd embodiment of the first aspect, the reaction is an antigen-antibody reaction wherein different antigen species are attached to various reaction zones of the plurality of reaction zones, and each reaction zone contains a single antigen species.

In a 37th embodiment of the first aspect which is also an embodiment of the 31st, the 32nd, the 33rd, the 34th, the 35th and the 36th embodiment of the first aspect, the labeling zone is arranged on the link, fixed link or fin connected to the at least one well.

In a 38th embodiment of the first aspect which is also an embodiment of the 31st, the 32nd, the 33rd, the 34th, the 35th and the 36th embodiment of the first aspect, the labeling zone is arranged on a rim of the multiwell plate.

In a 39th embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th, the 21st, the 22nd, the 23rd, the 24th, the 25th, the 26th, the 27th, the 28th, the 29th, the 30th, the 31st, the 32nd, the 33rd, the 34th, the 35th, the 36th, the 37th and the 38th embodiment of the first aspect, the first delivery device and the second delivery device are different.

In a 40th embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th, the 21st, the 22nd, the 23rd, the 24th, the 25th, the 26th, the 27th, the 28th, the 29th, the 30th, the 31st, the 32nd, the 33rd, the 34th, the 35th, the 36th, the 37th and the 38th embodiment of the first aspect, the first delivery device and the second delivery device are the same or of the same type.

In a 41st embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th, the 21st, the 22nd, the 23rd, the 24th, the 25th, the 26th, the 27th, the 28th, the 29th, the 30th, the 31st, the 32nd, the 33rd, the 34th, the 35th, the 36th, the 37th, the 38th, the 39th and the 40th embodiment of the first aspect, the first delivery device and the second delivery device are independently from each other selected from the group comprising ink jet printer, needle plotter and laser.

In a 42nd embodiment of the first aspect which is also an embodiment of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th, the 21st, the 22nd, the 23rd, the 24th, the 25th, the 26th, the 27th, the 28th, the 29th, the 30th, the 31st, the 32nd, the 33rd, the 34th, the 35th, the 36th, the 37th, the 38th, the 39th, the 40th and the 41st embodiment of the first aspect, the device is selected from the group comprising microarray, biosensor, lateral flow device.

The problem underlying the present invention is solved in a second aspect which is also the first embodiment of the second aspect, by a method for the manufacture or making of a device according to any one of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th, the 21st, the 22nd, the 23rd, the 24th, the 25th, the 26th, the 27th, the 28th, the 29th, the 30th, the 31st, the 32nd, the 33rd, the 34th, the 35th, the 36th, the 37th, the 38th, the 39th, the 40th, the 41st and the 42nd embodiment of the first aspect, comprising:
providing a device comprising a first surface and a second surface, at least one reaction zone and at least one labeling zone, wherein
   the at least one reaction zone is arranged on the first or the second surface and the at least one labeling zone is arranged on the first or the second surface,
   the at least one reaction zone allows performing a reaction, preferably a chemical, physical, biochemical or biological reaction, and
   the at least one labeling zone contains a marker or allows arranging a marker therein, wherein the marker allows the identification of the device; and
arranging a marker to the at least one labeling zone

wherein

a first chemical compound is added to the at least one reaction zone using a first delivery device and

the marker is added to the at least one labeling zone prior, during or after the adding of the first chemical compound to the at least one labeling zone using a second delivery device.

In a second embodiment of the second aspect which is also an embodiment of the first embodiment of the second aspect, the marker is added by plotting, contact-free blotting.

In a third embodiment of the second aspect which is also an embodiment of the second embodiment of the second aspect, the contact-free blotting is selected from the group comprising ink jet technology and laser blotting technology.

In a fourth embodiment of the second aspect which is also an embodiment of the first, the second and the third embodiment of the second aspect, the first and the first delivery device and the second delivery device are different.

In a fifth embodiment of the second aspect which is also an embodiment of the first, the second, the third and the fourth embodiment of the second aspect, the first delivery device and the second delivery device are the same or of the same type.

In a sixth embodiment of the second aspect which is also an embodiment of the first, the second, the third, the fourth and the fifth embodiment of the second aspect, the first delivery device and the second delivery device are independently from each other selected from the group comprising ink jet printer, needle plotter and laser plotter.

In a seventh embodiment of the second aspect which is also an embodiment of the first, the second, the third, the fourth, the fifth and the sixth embodiment of the second aspect, the marker is arranged on the first surface.

In an eighth embodiment of the second aspect which is also an embodiment of the first, the second, the third, the fourth, the fifth, the sixth and the seventh embodiment of the second aspect, the marker is arranged on the second surface.

In a ninth embodiment of the second aspect which is also an embodiment of the first, the second, the third, the fourth, the fifth, the sixth, the seventh and the eighth embodiment of the second aspect, the marker is an alphanumerical code, a binary code, a bar code, a 2D code, a color code, a triplet code or a combination thereof.

The problem underlying the present invention is solved in a third aspect by the use of a device according to any one of the first, the second, the third, the fourth, the fifth, the sixth, the seventh, the eighth, the ninth, the tenth, the eleventh, the twelfth, the 13th, the 14th, the 15th, the 16th, the 17th, the 18th, the 19th, the 20th, the 21st, the 22nd, the 23rd, the 24th, the 25th, the 26th, the 27th, the 28th, the 29th, the 30th, the 31st, the 32nd, the 33rd, the 34th, the 35th, the 36th, the 37th, the 38th, the 39th, the 40th, the 41st and the 42nd embodiment of the first aspect as a diagnostic device.

The present inventor has surprisingly found that it is possible to generate a labeled device comprising a first surface and a second surface, at least one reaction zone and at least one labeling zone, wherein

the at least one reaction zone is arranged on the first or the second surface and the at least one labeling zone is arranged on the first or the second surface,

the at least one reaction zone allows performing a reaction,

the at least one labeling zone contains a marker, wherein the marker allows the identification of the device and wherein

the labeled device is obtained by a method for the preparation of the labeled device, wherein

a first chemical compound is added to the at least one reaction zone using a first delivery device and

the marker is added to the labeling zone prior, during or after the adding of the first chemical compound to the at least one labeling zone using a second delivery device wherein the at least one reaction zone is arranged in a plane forming a reaction plane, and the at least one labeling zone is arranged in a plane forming a labeling plane and wherein the reaction plane and the labeling plane are at least partially overlapping. This arrangement of the marker and the reaction zones allows for the first time that the marker and the reaction zones form an identification entity. Such identification entity links the marker and the reaction zones in a direct manner. Because of such identification entity there is no risk that a marker is allocated to reaction zones for which it is not intended. This is particularly evident in case the labeled device and more specifically the at least one reaction zone thereof is analysed or viewed again, typically as an image of the labeled device: The marker which allows the identification of the at least one reaction zone is part of the image of the labeled device and thus immediately identifies the at least one reaction zone and, respectively, the plurality of such reaction zones contained on the device.

It is within the present invention that the delivery device which adds the first chemical compound to the at least one reaction zone is actually the same delivery device which adds the marker to the at least one labeling zone. Accordingly, a single one of the delivery device is used. It is, however, also within the present invention that two or more of said delivery device are used. In such case, the term “the delivery device” refers to the genus of such delivery device, and actually one or more of said device is/are used for the adding of the first compound to the at least one reaction zone, in particular if there is a plurality or multitude of such reaction zones on the device, and one or more of said device is/are used for the adding of the marker to the at least one reaction zone, in particular if there is a plurality or multitude of such labeling zones on the device.

The labeled device can be produced in various ways within the more general technical teaching outlined herein as will be discussed in the following.

In accordance with the instant invention the marker is, in an embodiment, added to the at least one labeling zone prior to the adding of the first chemical compound to the at least one reaction zone. In an embodiment of this embodiment, in case the device comprises a plurality or multitude of reactions zones, the marker is added to the at least one labeling zone prior to the adding of the first chemical compound to each and any reaction zone of the plurality or multitude of the reaction zones. In case the device comprises more than one labeling zone, in an embodiment, the marker is added to each and any of the labeling zones prior to the adding of the first chemical compound to each and any reaction zone of the plurality or multitude of reaction zones. As preferably used herein, a plurality of reaction zones means more than one reaction zone, two reaction zones, three reaction zones, four reaction zones and so on. More preferably a plurality of reaction zone means eight reaction zones, twelve reaction zones, 48 reaction zones, 96 reaction zones, 384 reaction zones and multiples of 96 reaction zones.

In accordance with the instant invention the marker is, in an embodiment, added to the at least one labeling zone after the adding of the first chemical compound to the at least one reaction zone. In an embodiment thereof, in case the device comprises a plurality or multitude of reactions zones, the marker is added to the at least one labeling zone after the adding of the first chemical compound to each and any reaction zone of the plurality or multitude of the reaction zones. In case the device comprises more than one labeling zone, in an embodiment, the marker is added to each and any of the labeling zones after the adding of the first chemical compound to each and any reaction zone of the plurality or multitude of reaction zones.

Finally, it is also in accordance with the instant invention that the marker is, in an embodiment and in case the device comprises a plurality or multitude of reaction zones, added to the at least one labeling zone after the adding of the first chemical compound to more than one of the reaction zones. In case the device comprises more than one labeling zone, in an embodiment, the marker is added to at least one, but not all of the labeling zones prior to the adding of the first chemical compound to each and any reaction zone of the plurality or multitude of reaction zones. It will be understood by a person skilled in the art that for those embodiments where the device comprises a plurality or multitude of reaction zones and a plurality or multitude of labeling zones, any possible sequence of adding the marker to the labeling zones and of adding the first chemical compound to the at least one reaction zone can be realized. This includes an alternation of adding a marker to one or more of the labeling zones and adding the first chemical compound to one or more of the reaction zones.

As preferably used herein, a labeled device is a device to which a marker has been added, whereby such marker allows the identification of the device.

In an embodiment, the device comprises a support. In a more preferred embodiment, the support is a solid support. The support can be of any of a variety of organic or inorganic materials or combinations thereof, including, merely by way of example, plastics such as polypropylene or polystyrene; ceramic; silicon; (fused) silica, quartz or glass, which can have the thickness of, for example, a glass microscope slide or a glass cover slip; paper, such as filter paper; diazotized cellulose; nitrocellulose filters; nylon membrane; or polyacrylamide or other type of gel pad, e.g., an aeropad or aerobead, made of an aerogel, which is, e.g., a highly porous solid, including a film, which is prepared by drying of a wet gel by any of a variety of routine, conventional methods. Supports that are transparent to light are useful when the method of performing an assay involves optical detection. In a preferred embodiment, the support is a multi-well plate, e.g., tissue culture dish, for example a 24-, 96-, 256-, 384-, 864- or 1536-well plate. Anchors can be associated, e.g., bound, directly with a surface, or can be associated with one type of support, e.g., glass, which in turn is placed in contact with a second support, e.g., within a plastic “well” in a microtiter plate. The shape of the support is not critical. It can, for example, be a flat support such as a square, rectangle, or circle; a curved support; or a three dimensional support such as a bead, particle, strand, precipitate, tube, sphere; etc.. In a more preferred embodiment, the support is selected from the group comprising glass slide, multi-well plate, membrane, lateral flow device and biosensor.

In an embodiment, the at least one reaction zone and the at least one labeling zone are arranged on the first surface. By arranging both the at least one reaction zone and the at least one labeling zone on the first surface the delivery device which adds the first chemical compound to the at least one reaction zone, can immediately add the marker to the at least one labeling zone without the need to change the relative position of the device to the delivery device. It will be understood by a person skilled in the art that if both the at least one reaction zone and the at least one labeling zone are arranged on the first surface, the at least one labeling zone and the at least one reaction zone are in essentially the same depth of focus. This is particularly given in case the device is a more two-dimensional device such as in case the device comprises as a support a glass slide. In an alternative embodiment the at least one reaction zone and the at least one labeling zone are not in the same focus level. This is particularly given in case the device is a more three-dimensional device such as in case the device comprises as a support a multi-well plate.

In an embodiment, in case the device comprises more than one labeling zone, the marker is added to each and any of the labeling zones prior to the adding of the first chemical compound to each and any reaction zone of the plurality or multitude of reaction zones. By arranging the at least one reaction zone on the first surface and the at least one labeling zone on the second surface, the delivery device which adds the first chemical compound to the at least one reaction zone, has to change its relative position to the device. However, in case two delivery devices of the delivery device which delivers the first chemical compound to the at least one reaction zone are used, one of said two delivery devices adds the first chemical compound to the at least one reaction zone on the first surface, and the other of said two delivery devices adds the marker to the at least one labeling zone on the second surface. It will also be understood by a person skilled in the art that in case two such delivery devices are used, the adding of the marker to the at least one labeling zone on the first surface by a first of said two delivery devices may be performed independently from the adding of the first chemical compound to the at least one reaction zone by a second of said two delivery devices. In any case, however, the marker is added to the at least one labeling zone on the second surface prior, during or after the adding of the first chemical compound to the at least one reaction zone on the first surface.

It will be understood that it is within the present invention that the at least one reaction zone and the at least one labeling zone are overlapping. The overlapping may be partial or complete. It is also within the present invention that in case there are more than one reaction zones and/or more than one labeling zones, some of the reaction zones overlap with some of the labeling zones. If both the at least one reaction zone and the at least one labeling zone are overlapping, the readout of the at least one reaction zone and any readout of the at least one labeling zone do not interfere. Also, the marker does not interfere with the activities of the at least one reaction zone. In case the at least one reaction zone and the at least one labeling zone are overlapping it is advantageous that the at least one reaction zone is arranged on the first surface of the device and the at least one labeling zone is arranged on the second surface of the device.

It is, however, also within the present invention that the at least one reaction zone of the device and the at least one labeling zone of the device are not overlapping. This arrangement of the at least one reaction zone and the at least one labeling zone is advantageous insofar that due to their spatial separation no interference occurs between the at least one labeling zone and the at least one reaction zone, or if such interference exists or might come into being it is less pronounced compared to a scenario of overlapping labeling zone(s) and reaction zone(s) which allows more levels of freedom as to the reaction performed in the at least one reaction zone and the at least one labeling zone, and the reading out of such reaction within the reaction zone and the marker in the labeling zone.

According to the present invention at least one reaction zone is arranged in a plane forming a reaction plane, and the at least one labeling zone is arranged in a plane forming a labeling plane, whereby it is preferred that the reaction plane and the labeling plane are at least overlapping. This arrangement is particularly advantageous with regard to reading out the reaction of the reaction zone and its result, respectively, and the reading of the marker. As preferably used herein, reading means, in a first step, detecting, and in a second optional step, transmitting what has been detected. In said first step, the reaction and the result thereof, respectively, as well as the marker are detected by a detection device. Such detection and detection device, respectively, can be combined or separate and independent from each other. In an embodiment, the detection device is a camera, a microscope, a combination of microscope and camera, a magnifying glass, a flat-bed scanner or the naked eye of an observer such as a human being a laser-scanner which is preferably used if the marker is or comprises a fluorescent dye, or a Geiger-Müller counter if the marker is or comprises a radiolabeled compound. In said second step, what has been detected by the detection device is transmitted from the detection device to an analysis device such as a computer where the results are analysed and optionally further processed. Further steps may follow. At the end of these steps, preferably, an information is provided to a user such as a human being as to which reaction occurred at which position of the device, what the result of such reaction is and/or what kind of conclusion may be drawn from the result.

In an embodiment of the labeled device of the invention the first detection device and the second detection device are different devices. In an alternative embodiment of the labeled device of the invention, the first detection device and the second detection device are the same or of the same type. The same applies to each and any of the other aspects of the invention.

Arranging the at least one reaction zone is a reaction plane and the at least one labeling zone in a labeling zone, allows for an easy handling of the at least one labeling zone and the at least one reaction zone. Among others, if the device comprises a plurality or multitude of reaction zones and optionally also a plurality or multitude of labeling zones and the reaction zones of the plurality or multitude of reactions zones are all or a part thereof arranged in a single plane, such arrangements allows for a convenient reading of the results of any reaction which occurs or has occurred in said reaction zones. More specifically, the reading can be made by a detection device the depth of focus does not have to be adapted between the reading of the individual reaction zone of the plurality of the reaction zones. The same is equally true if the device comprise additionally or alternatively a plurality or multitude of labeling zones and the labeling zones of the plurality or multitude of labeling zones are all or a part thereof arranged in a single plane. It is particularly advantages if the reaction plane(s) and the labeling zone(s) is/are overlapping as this means that the depth of focus of the detection means does not have to be changed which is particularly advantageous in the device is used in high-throughput procedures.

It will be understood that the kind of reaction which is performed in the at least one reaction zone or which is to be performed in said at least one reaction zone, are factually not limited. Any chemical, physical, biochemical or biological reaction which can be performed on a device described herein shall be encompassed by the instant invention. In an embodiment, the reaction is an interaction between at least the first chemical compound and a second chemical compound. In a preferred embodiment, the first chemical compound is attached to the at least one reaction zone. Such attachment can be either through covalent or non-covalent bonding. Also, the first chemical compound is either directly or indirectly attached to the at least one reaction zone. A direct attachment is typically achieved by one moiety of the first chemical compound being attached to the reaction zone. An indirect attachment is achieved by one moiety of the first chemical compound being attached to the at least one reaction zone through a linker. Such linker may be either provided by the at least one reaction zone or by the first chemical compound. Reactions for the attachment of the first chemical compound are known to a person skilled in the art.

The first chemical compound is added to the at least one reaction zone by a delivery device. Such delivery device is any device known in the art with which the first chemical reaction can be added to the at least one reaction zone, whereby said first chemical compound becomes attached to said at least one reaction zone upon having contacted said at least one reaction zone as described above. Suitable delivery devices as suitable for the practicing of the instant invention are those devices which are used in the art for the preparation of microarrays. Accordingly such delivery device is one selected from the group comprising a needle printer, an inject printer and a laser. Insofar, the first chemical compound may be added to the at least one reaction zone by contact methods using, among others a needle printer, or non-contact methods using, among others, an ink jet printer.

In the embodiment where a needle printer is used, the first chemical compound is attached to the at least one reaction zone by contacting a needle or pin of the needle printer with the at least one reaction zone. Attached to the needle is the first chemical compound. Upon contact of the needle with the at least one reaction zone the first chemical compound is transferred to the at least one reaction zone where the first chemical compound becomes attached to the at least one reaction zone. This technology is known to the person skilled in the art.

In the embodiment where an ink jet printer is used, the first chemical compound is attached to the at least one reaction zone such that the first chemical compound is contained in a fluid and such fluid is ejected from a printer, typically a printer nozzle. Upon contact of the fluid with the at least one reaction zone the first chemical compound is transferred to the at least one reaction zone where the first chemical compound becomes attached to the at least one reaction zone. In an embodiment thereof, the fluid is a liquid. Preferably such liquid is selected from the group comprising water, water-based solutions, alcoholic solutions, and buffers. It will be appreciated by the persons of the art that the fluid can be selected based on the chemical nature of the first chemical compound and the chemical nature of the at least one reaction zone. This technology is known to the person skilled in the art.

In the embodiment where the device of the invention comprises a plurality or multitude of reaction zones, and, optionally, also a plurality or multitude of labeling zones, the first chemical compound is preferably different for various of the plurality or multitude of reaction zones as is customary in the field of microarrays.

In an embodiment of the device of the invention, the first chemical compound is selected from the group comprising nucleic acid molecules, LNAs, peptides, polypeptides and proteins, antibodies, and carbohydrates, antigens such as allergens including but not limited to extracts from substances causing an allergy, cells (in case of cell-arrays) and tissues (in case of tissue arrays).

The second chemical compound is preferably a compound which is capable of interacting with the first chemical compound. In accordance with the principles underlying microarrays and the technology related thereto the microarray may be used to assess whether such second chemical compound is present in a sample. Such sample may be any sample such as a sample for diagnostic purposes, a sample from basic research, a sample from a production process and the like. It is within the present in invention that the sample which may or may not contain the second chemical compound is added to the at least one reaction zone. In the embodiment where the device of the invention comprises a plurality or multitude of reaction zones, the sample may be added to one or several of the plurality or multitude of reaction zones individually. Alternatively, the sample is added to the device of the invention as a whole and, accordingly, the sample is not added individually to one or several of the plurality or multitude of reaction zones.

In an embodiment of the device of the invention the marker is an identification code. Such marker and identification code allows the identification of the device. Once the device such as a microarray is identified, any information related to such device can be retrieved, for example from a databank which contains such information. Such information can, among others, be related to what kind of first chemical compound has been added to the at least one reaction zone, or, in case the device comprises a plurality or multitude of such reaction zones, which first chemical compound is attached to which one of said plurality or multitude of reaction zones, the production date, the production conditions, the intended use, the intended user, the orientation of the device necessary for the read out of the device etc.

The marker of the device of the invention is present as or comprises a code, whereby such code is selected from the group comprising an alphanumerical code, a binary code, a bar code, a 2D code, a color code, a triplet code or a combination thereof. In a preferred embodiment the code is a binary code which is arranged in the at least one labeling zone of the device of the invention. It is within the invention that the entire code is arranged in one labeling zone of the device of the invention. It is, however, also within the invention that, in case the device of the invention comprises a plurality or multitude of labeling zones, the marker is contained in more than one of the labeling zones. It is also within the invention that various parts of the marker are contained in various of the labeling zones of the plurality or multitude of labeling zones, whereby, preferably, the entire marker is contained in the various of the labeled zones so that upon putting together the parts of the marker which are spread over the various of the labeling zones of the plurality of the labeling zones, the entire maker is obtained and reconstituted, respectively.

In a preferred embodiment, the marker is a binary marker. Using a binary marker consisting of, for example, 16 bits allows the identification of 65.536 devices (2¹⁶). It is within the present invention that the if the binary code is used an individual bit of such binary code is represented by and thus contained in an individual labeling zone of a device of the invention comprising a plurality of labeling zones.

The marker as such can be prepared by using the delivery device as described herein. It is within the present invention that the marker is prepared from the first chemical compound. It is, however, also within the present invention that the marker is prepared from one or more than one compounds which are different from the first chemical compound. In a preferred embodiment, the marker is prepared from a compound which provides a signal which generates the marker or provides an image thereof, under conditions where the interaction between the first chemical compound and the second chemical compound occurs and/or conditions where the result of the reaction between the first chemical compound and the second chemical compound can be detected. The following are illustrative examples. If the first chemical compound is a nucleic acid which interacts with the second chemical also being a nucleic and the reaction between the first chemical compound and the second chemical compound can be monitored and detected, respectively, by fluorescence or a hybridization signal. In this embodiment, the marker could advantageously also be a nucleic acid which could be identified by the same means, i.e. fluorescence or hybridization. In another example, the first chemical compound is an antigen and the second chemical compound is a human antibody. Such human antibody is detected by using an anti-human antibody. In this embodiment the marker can be formed by a human antibody and the marker can accordingly be detected by using an anti-human antibody. The anti-human antibody used for the detection of the marker formed by a human antibody can be either the anti-human antibody which is used for detecting the human antibody bound to the antigen forming the first chemical compound, or an anti-human antibody which is different therefrom.

In another embodiment of the device of the invention the marker consist of a dye including but not limited to black ink, blue ink, fluorescent ink and radioactive ink. In a further embodiment the marker is or comprises a biological compound, wherein such biological is selected from the group comprising a nucleic acid molecule, an oligonucleotide, a DNA oligonucleotide, an RNA oligonucleotide, an LNA oligonucleotide, a biotinylated oligonucleotide, a biotinylated DNA oligonucleotide, a biotinylated RNA oligonucleotide, a biotinylated LNA oligonucleotide, a color-labeled oligonucleotide, a color-labeled DNA oligonucleotide, a color-labeled RNA oligonucleotide, a color-labeled LNA oligonucleotide, a protein, an oligopeptide, a peptide, an antibody, a biotinylated protein, a biotinylated oligopeptide, a biotinylated peptide, a biotinylated antibody.

The device according to the present invention is, in one embodiment, a slide wherein the slide contains a plurality of reaction zones. This embodiment, i.e. a slide based device, is customary in the field of microassays. In a preferred embodiment the reaction zones are each and individually a hydrophilic anchor surrounded by a hydrophobic zone. In a further embodiment thereof, also the at least one labeling zone is such a hydrophilic anchor. This kind of slides and microarrays, respectively, are described in European patent application EP 1230013.

The device according to the present invention is, in one embodiment, multi-well plate comprising a plurality of wells which are connected through links. In this embodiment an individual well of the multi-well plate forms a device of the invention with all the embodiments thereof as described herein. It is, however, also within the present invention that an individual well forms a reaction zone of the device of the invention.

In those embodiments of the invention where the device of the invention is a multi-well plate the at least one labeling zone is contained in at least one of the wells of the multi-well plate. Preferably the at least one labeling zone is arranged on the same surface as the at least one reaction zone, which is preferably arranged on the bottom of the well. In another embodiment thereof, the at least one labeling zone is arranged on a rim of the multi-well plate, whereby such rim connects one or several of the wells of the multi-well plate. It is within the present invention that the device comprises a plurality or multitude of labeling zones and that some or all of the rims of the multi-well plate form or contain a labeling zone. It is within the present invention that the marker is contained in a single labeling zone or that parts of the marker are contained in various of the labeling zones, whereby the entire marker is obtained when combining or putting together the various labeling zones.

The method of invention for the manufacture or making of the device of the invention makes use of the same technology and steps as described in connection with the device of the invention which are insofar incorporated herein by reference.

Methods for the manufacture of DNA chips which, when comprising a marker as defined herein, are an embodiment of the labeled device of the present invention are among others described by Pirrung M. C (Pirrung M. C, Angew. Chem. Int. Ed. 2002, 41, 1276-1289)

The present invention is now further illustrated by the attached FIGURES from which further features, embodiments and advantages may be taken.

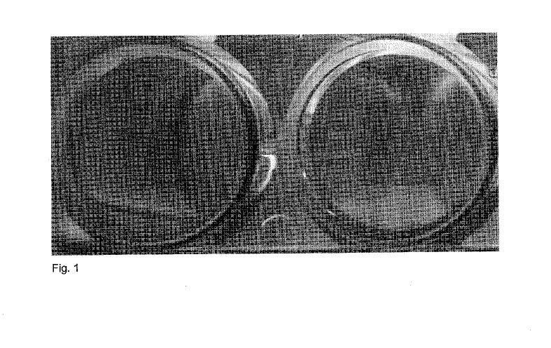

More specifically, FIG. 1 shows photographs of two embodiments of the device of the invention.

FIG. 1 is a photograph of two devices of the invention.

In the following, the device depicted on the left side of FIG. 1 is described in more detail.

The displayed device of the invention is a multi-well plate. At the bottom of a single well a microarray has been printed by means of an ink-jet printer (sciFLEXARRAYER). The microarray consists of a two dimensional matrix of 30 dots for each dimension.

Each dot of the 30×30 matrix represents a reaction zone of the device of the invention. Into each of the reaction zones a biotinylated DNA oligonucleotide has been printed by the ink-jet The biotinylated DNA oligonucleotide has been incubated with a conjugate of streptavidine and peroxidase. Upon addition of a TMB derivative the TMB derivative reacts with the peroxidase which provides a colored precipitate which may then be detected and allows the identification of the individual reaction zones.

Above the two dimensional matrix of reaction zones a labeling zone is arranged. Into said labeling zone, an identification code has been added as a marker which allows the identification of the device. Such marker consist of a 2D code and more specifically of a 25 dot code. Such 25 dot code (arranged either in a row or as a 5×5 matrix as in the embodiment subject to FIG. 1 allows the encoding of 2²⁵ arrays. The marker was added to the labeling zone also by an ink jet printer and visualized by the same technique as the individual reaction zones of the 30×30 matrix In the embodiment of the device of the invention depicted on the left side of FIG. 1 the first surface bears both the reaction zones and the labeling.

The depicted device of the invention allows that the information provided by the marker is retrieved from the device at the same time when the result of the reactions in the individual reaction zones of the device are retrieved. Because of this an immediate and one to one correlation between the device actually used and the result(s) obtained therefrom is possible.

The device depicted on the right side of FIG. 1 is factually the same as the one displayed on the left side of FIG. 1 and has basically been prepared the same way. However, in the embodiment of the device of the invention depicted on the right side of FIG. 1 the reaction zones are present on a first surface of the device which is in the instant case the inner bottom of the well, whereas the labeling zone is present on a second surface which is in the instant case the outer bottom of the well.

The features of the present invention disclosed in the specification, the claims and/or the drawings may both separately and in any combination thereof be material for realizing the invention in various forms thereof.

1-52. (canceled)

53. A labeled device comprising a first surface and a second surface, at least one reaction zone and at least one labeling zone, wherein
the at least one reaction zone is arranged on the first or the second surface and the at least one labeling zone is arranged on the first or the second surface,
the at least one reaction zone allows performing a reaction, preferably a chemical, physical, biochemical or biological reaction,
the at least one labeling zone contains a marker, wherein the marker allows the identification of the device and wherein
the labeled device is obtained by a method for the preparation of the labeled device, wherein
a first chemical compound is added to the at least one reaction zone using a first delivery device,
the marker is added to the at least one labeling zone prior, during or after the adding of the first chemical compound to the at least one labeling zone using a second delivery device,
wherein the at least one reaction zone is arranged in a plane forming a reaction plane, and the at
least one labeling zone is arranged in a plane forming a labeling plane and wherein the reaction plane and the labeling plane are at least partially overlapping.

54. The device according to claim 53, wherein the at least one reaction zone and the at least one labeling zone are arranged on the first surface.

55. The device according to claim 53, wherein the at least one reaction zone is arranged on the first surface and the at least one labeling zone is arranged on the second surface.

56. The device according to claim 53, wherein the at least one reaction zone and the at least on labeling zone are overlapping or are at least partially overlapping.

57. The device according to claim 53, wherein the at least one reaction zone and the at least one labeling zone are not overlapping.

58. The device according to claim 53, wherein the marker and the at least one reaction zone form an identification entity.

59. The device according to claim 53, wherein the reaction plane and the labeling plane are detectable at the same time.

60. The device according to claim 53, wherein the marker is an alphanumerical code, a binary code, a bar code, a 2D code, a color code, a triplet code or a combination thereof.

61. The device according to claim 53, wherein the marker is formed by an ink.

62. The device according to claim 53, wherein the device comprises a plurality of reaction zones.

63. The device according to claim 53, wherein the device is a multi-well plate comprising a plurality of wells which are connected through links.

64. The device according to claim 63, wherein at least one of the wells contains the plurality of reaction zones.

65. The device according to claim 53, wherein the first delivery device and the second delivery device are the same.

66. The device according to claim 53, wherein the device is selected from the group comprising microarray, biosensor, lateral flow device.

67. A method for the manufacture or making of a device, comprising:
providing a device comprising a first surface and a second surface, at least one reaction zone and at least one labeling zone, wherein
the at least one reaction zone is arranged on the first or the second surface and the at least one labeling zone is arranged on the first or the second surface,
the at least one reaction zone allows performing a reaction, preferably a chemical, physical, biochemical or biological reaction, and
the at least one labeling zone contains a marker or allows arranging a marker therein, wherein the marker allows the identification of the device; and
arranging a marker to the at least one labeling zone
wherein
a first chemical compound is added to the at least one reaction zone using a first delivery device and
the marker is added to the at least one labeling zone prior, during or after the adding of the first chemical compound to the at least one labeling zone using a second delivery device.

68. The method according to claim 67, wherein the marker is added by plotting, contact-free blotting.

69. The method according to claim 68, wherein the contact-free blotting is selected from the group comprising ink jet technology and laser blotting technology.

70. The method according to claim 67, wherein the first delivery device and the second delivery device are the same.

71. The method according to claim 67, wherein the marker is an alphanumerical code, a binary code, a bar code, a 2D code, a color code, a triplet code or a combination thereof.

72. The method according to claim 67, wherein the marker is formed by an ink.
